(19) United States
(12) Patent Application Publication
(10) Pub. No.: US 2024/0269151 A1
(43) Pub. Date: Aug. 15, 2024

(54) NASAL SLEEP FORMULATION

(71) Applicant: CS MEDICA A/S, Copenhagen (DK)

(72) Inventor: Lone HENRIKSEN, Fredensborg (DK)

(21) Appl. No.: 18/289,726

(22) PCT Filed: May 3, 2022
(86) PCT No.: PCT/EP2022/061792

§ 371 Date: Nov. 6, 2023

(30) Foreign Application Priority Data
May 4, 2021 (DK) PA 2021 70207

(51) Int. Cl. A61K 31/00; A61K 9/00; A61K 31/355; A61K 36/53; A61P 25/00

(52) U.S. Cl. CPC A61K 31/658; A61K 9/0043; A61K 9/0073; A61K 31/355; A61K 36/53; A61P 25/00

(57) ABSTRACT

A composition for treatment of sleep disorders is disclosed, said composition comprising by weight: 5-30% hemp oil: 0.1-5.0% cannabidiol (CBD) and/or cannabinol (CBN): 0.01-1.0% lavender oil: 30-95% (or up to 100%) sesame oil: and 0.1-5.0% vitamin E and/or Tocopherol equivalents. Such a composition can e.g. be administered intra-nasally, such as by the use of a nasal spray bottle.

### FIELD OF THE INVENTION

The present invention relates to a composition formulated for intranasal application, such as a nasal spray for use in the treatment and/or alleviation of sleeping- or relaxation-related conditions or disorders in a subject. Treatments may comprise nasal application of a cannabinoid-comprising composition, such as a cannabinol—(CBN) and cannabidiol—(CBD) comprising an oil-based composition.

### BACKGROUND OF THE INVENTION

WO 2019106652 concerns CBD-comprising compositions for treating neurological disorder, muscular disturbances, ticks and insomnia.

WO18232448 concerns sleep disorder compositions and treatment thereof and discloses compositions for treating sleep comprising THC and further cannabinoids.

WO2019003163 concerns terpene-enriched cannabinoid product for women health.

US20190314326 discloses dilutable formulations of cannabinoids and processes for their preparation.

US20190183849 pertains to compounds and methods for treatment of disease and disorders, and discloses compositions comprising tetrahydrocabinol (THC) and further cannabinoids.

Insomnia and other sleep disorders are a general problem worldwide, and a significant proportion of the population suffers from sleeping disorders, as well as thereto related conditions and problems. Sleep disorders can interfere with normal physical, mental, social and emotional functioning of a subject, and are thus severe.

Common treatment of sleep disorders comprises e.g. sleeping pills, melatonin supplements, allergy or cold medication, medications for any underlying health issues breathing device or surgery (usually for sleep apnoea), a dental guard (usually for teeth grinding).

There is a need for compositions and/or treatments for insomnia and other sleep disorders.

### SUMMARY OF THE INVENTION

As presented herein, surprisingly and/or unexpectedly, and from a wide range of component candidates and concentration ranges, the inventors have found the following compositions to be effective in relation to treatment and/or alleviating of conditions and symptoms related to sleep disorder(s) in a subject. Furthermore, such formulations and their methods of administration are also believed to be useful in the treatment or amelioration of conditions related to e.g. Parkinson, multiple sclerosis (MS), muscle spasmed, anxiety, depression, Alzheimer, epilepsy, pain, and/or conditions or diseases requiring a neuroprotective effect.

In a first aspect, the present invention concerns a composition comprising by weight: 5-30% hemp oil; 0.1-5.0% cannabidiol (CBD) and/or cannabinol (CBN); 0.01-1.0% lavender oil; 30-95% (or up to 100%) sesame oil; and 0.1-5.0% vitamin E and/or Tocopherol equivalents. The composition can be formulated for nasal application, e.g. as nasal spray. Compositions are disclosed, comprising e.g. 5-30%, 10-20%, or ˜16% hemp oil; 0.1-5.0%, 0.2-2.0%, or ˜0.4% CBD and/or 0.1-5.0%, 0.2-2.0%, or ˜0.8% cannabinol CBN; 0.01-1.0%, 0.02-0.5%, or ˜0.03% lavender oil; 30-95%, 50-90%, or ˜82% sesame oil; and/or 0.1-5.0%, 0.2-1.0%, or ˜0.55% vitamin E and/or Tocopherol equivalents).

In some embodiments the CBD used in the provision of the composition is crystalline, such as “type A CBD” as disclosed herein. In some embodiments, said CBD is provided as—or capable of forming—needle-like crystals.

In a second aspect, the present invention relates to a method for providing a composition, such as a composition for nasal application according to the first aspect. Such a method may comprise the steps or acts of (i) providing hemp oil comprising CBD and/or CBN; (ii) providing lavender oil, sesame oil and vitamin E (e.g. vitamin E oil); and (iii) mixing the hemp oil from step (i) with the ingredients from step (ii); and optionally (iv) aliquoting the composition of step (iii) into one or more receptacle(s), such as nasal pump spray bottle(s) adapted to provide 50-350 μl, 100-250 μl, or around 160 μl per “puff” (=volume of (liquid) composition for nasal application which is dispersed when activating a pump spray once).

In a third aspect, the present invention pertains to a composition provided by a method according to the second aspect.

In a fourth aspect, the present invention concerns a receptacle comprising a composition according to the first, third, seventh or eighth aspect, such as a nasal pump spray bottle.

In a fifth aspect, the present invention relates to a kit comprising a receptacle according to the fourth aspect, and optionally, comprising an instruction for use.

In a sixth aspect, the present invention pertains to a method for treatment of a subject, comprising intranasal application or administration of a composition according to the first, third, seventh or eighth aspect.

In a seventh aspect, the present invention concerns a composition according to the first, third, or eighth aspect for use as a medicament and/or therapeutic agent. This may comprise treatment of one or more sleep disorder(s), such as and/or related to one or more of: Insomnia; Snoring; Obstructive Sleep Apnoea; Sleep Hypoventilation; Restless Legs Syndrome; Bruxism; Narcolepsy; Sleep talking, sleep walking and/or other automatic behaviours; Nightmares and/or night terrors; and/or Rapid eye movement behaviour disorder. Further conditions and/or diseases are disclosed herein.

In an eighth aspect, the present invention pertains to a pharmaceutical composition comprising or consisting essentially of a composition according to the first, third or seventh aspect, optionally comprising one or more pharmaceutically acceptable carrier(s) and/or diluent(s).

In a ninth aspect, the present invention concerns a CBD-comprising composition, such as an intranasal composition, wherein the CBD used in the formulation is crystalline and/or “type A CBD”. In some embodiments, said composition is a composition as disclosed in the first, third, seventh or eighth aspect. In some embodiments, the CBD is of type A (e.g. needle-like crystals) and/or capable of forming needle-like crystals. as disclosed herein, e.g. in the first aspect and/or Examples.

In a tenth aspect, the present invention pertains to a dosage regimen, comprising administering a composition, such as a CBD- and/or CBD comprising composition such as an intra-nasal composition as disclosed herein, e.g. according to the first, third, seventh, eighth or ninth aspect. In some embodiments, the CBD is of “type A”.

### DESCRIPTION OF THE DRAWINGS/FIGURES

FIG. 1: microscope picture of cannabinol (CBD) forming needle-like crystals. The CBD crystals were sourced from www.enecta.com.

FIG. 2: microscope picture of cannabinol (CBD) forming cluster- or bunch-like crystals. The CBD crystals were sourced from www.pharma-hemp.com.

### DETAILED DESCRIPTION OF THE INVENTION

### Definitions

In the context of the present invention, the singular form of a word may include the plural, and vice versa, unless the context clearly dictates otherwise. Thus, the references “a,” “an” and “the” are generally inclusive of the plurals of the respective terms. For example, reference to “an ingredient” or “a method” may include a plurality of such “ingredients” or “methods.”

Similarly, the words “comprise,” “comprises,” and “comprising” are to be interpreted inclusively rather than exclusively. Embodiments provided by the present disclosure may lack any element that is not specifically disclosed herein. Thus, a disclosure of an embodiment defined using the term “comprising” is also a disclosure of embodiments “consisting essentially of” and “consisting of the disclosed components”. Thus, the term “comprising” is generally to be interpreted as specifying the presence of the stated parts, steps, features, or components, but does not exclude the presence of one or more additional parts, steps, features, or components. For example, a composition comprising a chemical compound may thus comprise additional chemical compounds.

Generally, compositions as disclosed herein, in particular topical compositions and/or compositions for oral consumption may comprise one or more pharmaceutically acceptable carrier(s), excipient(s), stabilizer(s) or the like.

Where used herein, terms like “for example”, “e.g.” or “such as”, particularly when followed by a listing of terms, is merely exemplary and illustrative, and should not be deemed to be exclusive or comprehensive. Any embodiment disclosed herein may be combined with any other embodiment disclosed herein.

Unless expressed otherwise, all percentages expressed herein are by weight of the total weight of the composition. Thus, unless indicated otherwise, “%” indicates “% weight/weight (w/w)”, also called “weight %” or “% by weight”.

In the context of the present invention, the terms “about”, “around”, “approximately” or the symbol “˜” can be used interchangeably, and are meant to comprise variations and/or uncertainties generally accepted in the field, e.g. comprising analytical errors and the like. Thus “about” may also indicate measuring uncertainty commonly experienced in the art, which can 10 be in the order of magnitude of e.g. +/−1, 2, 5, 10, or even 20 percent (%). Furthermore, “about” may be understood to refer to numbers in a range of numerals, for example the range of +/−20, +/−15, +/−10, +/−5, +/−2, +/−1, +/−0.5, +/−0.1% of the referenced number. Moreover, all numerical ranges herein should be understood to include all integers, whole or fractions, within the range.

As used herein, the term “in some embodiments” is meant to comprise “in one embodiment”, “in some embodiments”, and “in one or more embodiments”.

In the context of the present invention, the terms “subject” or “patient” can be used interchangeably, and are meant to comprise a human, animal and/or mammal. In particular, a human subject can e.g. be selected from one or more of: female, male, senior, adult, adolescent, child, or infant. An animal subject can e.g. be selected from pet, husbandry, mammal, reptile, bird, and/or animal in a zoo.

In the context of the present invention, the term “treatment” is meant as an act aiming at alleviating, lessen, improving and/or curing any symptom(s), condition(s), or disease(s) in a subject. The effect or efficiency of a treatment can be assessed by a control, e.g. no treatment, treatment with a known composition, or treatment with a placebo. Generally, a “treatment” in the present context comprises administration of a suitable amount of a composition to a subject. Compositions of the present invention are preferably administered intra-nasally, such as by a nose spray using a nasal pump spray device used in the treatment of nasal congestion, such as Navision® or Otrivin® comprising xylometazoline, such as xylometazoline hydrochloride.

Alternatively, the composition can be administered inside the nostril(s) by other means, such as applying a suitable amount with e.g. a finger or applicator, and/or by “sniffing” or “snorting”.

In the context of the present invention, the terms “intra-nasal”, “intranasal” and simply “nasal” can be used interchangeably. This applies as well for “intra-nasally”, “intranasally” and simply “nasally”.

A “sleep disorder” or “somnipathy” can be described as a disorder of the sleep patterns of a subject. Sleep disorders are common in both children and adults. Disruptions in sleep can be caused by a variety of issues, including teeth grinding (bruxism) and night terrors. Commonly, when a subject suffers from difficulty falling asleep and/or staying asleep with no obvious cause, it is referred to as insomnia. Sleep disorders can e.g. be classified into dyssomnias, parasomnias, circadian rhythm sleep disorders involving the timing of sleep, and other disorders including ones caused by medical or psychological conditions.

The most common sleep disorder is insomnia. Other disorders are sleep apnoea, narcolepsy and hypersomnia (excessive sleepiness at inappropriate times), sleeping sickness (disruption of sleep cycle due to infection), sleepwalking, and night terrors.

The risk of developing sleep disorders in the elderly is especially increased for sleep disordered breathing, periodic limb movements, restless legs syndrome, REM sleep behaviour disorders, insomnia and circadian rhythm disturbances.

Nasal sprays are used to deliver medications locally in the nasal cavities or systemically. They are used locally for conditions such as nasal congestion and allergic rhinitis. In some situations, the nasal delivery route is preferred for systemic therapy because it provides an agreeable alternative to injection or pills. Substances can be assimilated extremely quickly and directly through the nose. Many pharmaceutical drugs exist as nasal sprays for systemic administration (e.g. sedative-analgesics, treatments for migraine, osteoporosis and nausea). Other applications include hormone replacement therapy, treatment of Alzheimer's disease and Parkinson's disease. Nasal sprays are often seen as a more efficient way of transporting drugs with potential use in crossing the blood-brain barrier.

Apart from treatment of one or more sleep disorders, the present compositions are also believed to be suitable to provide relaxation of a subject, in particular nervousness or even fear, in particular irrational fear. Examples may comprise user situations in e.g. public transport, passengers in aircraft, train, bus, or car, such as transport situations, where the subject would like to relax and/or sleep, but is hindered to do so because of anxiety. Further conditions and/or examples may comprise jet lag, anxiety, and/or difficulties in falling asleep under unusual circumstances, e.g. when travelling, e.g. not sleeping at home, e.g. during transport in a bus, car, train, aeroplane, ship, hotel, pension, prison, or in a hospital, hospice, or the like, as well as during military service or the like. In some embodiments, the present composition provides a positive effect and/or treatment in a condition related to Parkinson, multiple sclerosis (MS), muscle spasmed, anxiety, depression, Alzheimer, epilepsy, pain relief, and/or neuroprotective effects.

In a first aspect, the present invention concerns a composition comprising by weight: 5-30% hemp oil; 0.1-5.0% cannabidiol (CBD) and/or cannabinol (CBN); 0.01-1.0% lavender oil; 30-95% (or up to 100%) sesame oil; and 0.1-5.0% vitamin E and/or Tocopherol equivalents.

The composition can be formulated for nasal application, e.g. as nasal spray.

“Hemp seed oil” or “hemp oil” is obtained by pressing hemp seeds. It is rich in healthful oils and fatty acids, is popular as a remedy for a range of conditions including skin issues and stress. It may also contain properties that contribute to reduced risks of illnesses like Alzheimer's disease and cardiovascular disease. Hemp oil may also reduce inflammation in the body. Hemp oil contains large amounts of omega-6 and omega-3 fats, and all nine essential amino acids. Hemp seeds also contain the following compounds: Vitamin C, Calcium, Iron, Omega-3 fatty acids, Gamma linolenic acid, Arginine, Magnesium, and B vitamins. Hemp oil may comprise further compounds, there among cannabinoids, but only in very low or trace amounts.

“Cannabidiol” or “CBD”, CAS no. 3956-29-1, is a commonly cannabinoid used to address a variety of medical conditions, including insomnia. In contrast to e.g. tetrahydrocarbinol (THC) and tetrahydrocannabivarin (THCV), CBD is considered to be one of the many non-psychoactive cannabinoids found in Cannabis.

“Cannabinol” or “CBN”, CAS no 521-35-7, is a cannabinoid that has been shown to help effectively as a sleep aid or sedative, to regulate the immune system and works to relieve the pain and inflammation caused by several conditions, including insomnia, arthritis and Crohn's disease. It is believed to be mildly psychoactive, and only found in trace amounts in Cannabis.

“Lavender essential oil” or “lavender oil” is used e.g. in aromatherapy. Commonly, lavender oil is produced by steam distillation of Lavandula angustifolia flowers. The oil promotes relaxation and is believed to treat anxiety, fungal infections, allergies, depression, insomnia, eczema, nausea, and menstrual cramps.

“Sesame oil” is an edible oil provided by pressing sesame seeds. Sesame oil can also be used for a variety of treatments and ailments. Sesame oil is believed to possess antifungal, antiviral, as well as anti-inflammatory properties. Furthermore, it contains antioxidants such as sesamol and sesaminol.

“Vitamin E” is a group of eight fat soluble compounds that include four tocopherols and four tocotrienols. Both the tocopherols and tocotrienols occur in α-, β-, γ-, and δ-forms, as determined by the number and position of methyl groups on the chromanol ring. Vitamin E is the major lipid-soluble antioxidant in the cell antioxidant defense system and is exclusively obtained from the diet. Vitamin E has health promoting properties that are attributed to its antioxidant action and its ability to stabilize cell membrane and promote restoration of the skin barrier function. Commonly, vitamin E activity of the different vitamin E isomers is expressed in “α-tocopherol equivalents”, or simply “tocopherol equivalents” (“TE”) herein. One TE is the activity of 1 mg RRR-α-tocopherol (d-α-tocopherol). According to the Food and Agriculture Organisation of the United Nations, e.g. β-tocopherol should be multiplied by 0.5, γ-gamma-tocopherol by 0.1, and a-tocotrienol by 0.3.

In some embodiments, the composition comprises 5-30%, 10-20%, or ˜16% hemp oil.

In some embodiments, the composition comprises 0.1-5.0%, 0.2-2.0%, or ˜0.4% CBD.

In some embodiments, the composition comprises 0.1-5.0%, 0.2-2.0%, or ˜0.8% CBN.

In some embodiments, the composition comprises 0.01-1.0%, 0.02-0.5%, or ˜0.03% lavender oil.

In some embodiments, the composition comprises 30-95%, 50-90%, or ˜82% sesame oil.

In some embodiments, the composition comprises 0.1-5.0%, 0.2-1.0%, or ˜0.55% vitamin E and/or Tocopherol equivalents).

Thus, in some embodiments a composition is provided comprising by weight:
i. 5-30%, 10-20%, or ˜16% hemp oil;
ii. 0.1-5.0%, 0.2-2.0%, or ˜0.4% cannabidiol (CBD); and/or 0.1-5.0%, 0.2-2.0%, or ˜0.8% cannabinol (CBN);
iii. 0.01-1.0%, 0.02-0.5%, or ˜0.03% lavender oil;
iv. 30-95%, 50-90%, or ˜82% sesame oil; and
v. 0.1-5.0%, 0.2-1.0%, or ˜0.55% vitamin E and/or Tocopherol equivalents.

Such compositions comprising CBD, CBN, or CBD and CBN can be, or are formulated for nasal application, such as for administering a suitable, preferably defined amount by appropriate means, such as by spraying, e.g. by the use of a nasal spray bottle. Generally, the composition will often be called “nasal spray composition”, or simply “nasal composition” herein; both terms can be used interchangeably.

In some embodiments, the nasal composition comprises both CBN and CBD. Surprisingly and unexpectedly, compositions comprising more CBN than CBD showed a better effect. Thus, in some embodiments, the CBN:CBD ratio by weight is greater than 1.

In some embodiments, CBN:CBD ratio can be in the range of 10:1-5:1; 5:1-4:1; 4:1-3:1; 3:1-2:1; or 2:1-1.1. In some embodiments, the CBN:CBD ratio is around 10:1, ˜9:1, ˜8:1, ˜7:1, ˜6:1, ˜5:1, ˜4:1, ˜3.5:1, ˜3:1, ˜2.5:1, ˜2:1, ˜1.5:1, ˜1.25:1, ˜1.2:1, ˜1.15:1 or ˜1.1:1. In some embodiments, the CBN:CBD ratio is in the range of 2.5:1- 1.5:1, 2.25:1.75, 2.2-1.8, 2.1-1.9, or around 2:1. In some embodiments, the CBN:CBD ratio is in the range of 5:1 to 1:1, 4:1 to 1.1:1, 3:1 to 1.1:1, 2.5:1 to 1.1:1, 2.2:1 to 1.1., 2 to 1.1, 1.75:1, 1.5:1, 1.25: 1, or 1.15 to 1. In some embodiments, the ratio of CBN to CBD of around 3:1-1.5:1, 2.5:1.75, 2.2-1.8, 2.1:1:9, or around 2:1.

Ratios of e.g. 3:1-1.5 are believed to provide a good balance of the two cannabinoids in terms of treatment of e.g. sleep disorders, and/or one or more of the other conditions disclosed herein.

In some embodiments, the nasal composition may comprise one or more further cannabinoid(s), such as one or more psychoactive and/or one or more non-psychoactive cannabinoid(s) in a physiologically active amount. Such a further cannabinoid may e.g. be selected from one or more of: THC (tetrahydrocannabinol), THCA (tetrahydrocannabinolic acid), CBDA (cannabidiolic acid), CBG (cannabigerol), CBC (cannabichromene), CBL (cannabicyclol), CBV (cannabivarin), THCV (tetrahydrocannabivarin), THCP (tetrahydrocannabiphorol), CBDV (cannabidivarin), CBCV (cannabichromevarin), CBGV (cannabigerovarin), CBGM (cannabigerol monomethyl ether), CBE (cannabielsoin), and CBT (cannabicitran), including any combination(s) thereof. In some embodiments, one or more cannabinoids are present in non-physiologically significant amounts, such as impurities in one or more of: hemp oil, CBN, and or CBD. Commonly, an impurity is present in less than 1.5, 1.0, 0.5, 0.2, or 0.1% of said one or more further cannabinoid by with respect to either: total composition, hemp oil, CBN, or CBD.

In some embodiments, a composition may thus comprise 0.1-5.0% CBN and/or CBD, wherein the composition, CBN, CBD, and/or hemp oil (x) does not comprise, (y) does not comprise in a physiologically active amount, and/or (z) comprises less than 2.0, 1.5, 1.0, 0.5, 0.2, or 0.1% (w/w) of one or more further cannabinoid(s), such as a one or more cannabinoids selected from THC, THCA, CBDA, CBG, CBC, CBL, CBV, THCV, THCP, CBDV, CBCV, CBGV, CBGM, CBE, and CBT.

Also, a low content of one or more of CBDV, CBDA, CBG, THC and/or THCV can be indicative of a CBD and/or CBN of sufficient purity for compositions of the present invention.

Suitable hemp oil(s), CBD, and/or CBN with sufficient purity can e.g. be sourced from www.enecta.com.

In some embodiments, CBD is “crystalline” or “pure” CBD, such as CBD in powder-form, with a purity of at least 98%, and comprising less than 1.5% (w/w) of CBDV, CBG, and/or CBN, and less than 0.05% THC.

In some embodiments, CBN is “crystalline” or “pure” CBN, such as CBN in powder-form, with a purity of at least 98%, and comprising less than 1.5% (w/w) of CBDV, CBG, and/or CBD, and less than 0.05% THC.

In some embodiments, the composition and/or hemp oil comprises ≤0.2% or ≤0.05% CBDV by weight. Generally, a low content of CBDV and/or other cannabinoids is desirable.

In some embodiments, the composition and/or hemp oil comprises ≤0.2% or ≤0.05% CBDA by weight. Generally, a low content of CBDA and/or other cannabinoids is desirable.

In some embodiments, the composition and/or hemp oil comprises ≤0.5% or ≤0.025% CBG by weight. Generally, a low content of CBG and/or other cannabinoids is desirable.

In some embodiments, the composition and/or hemp oil comprises ≤0.05% or ≤0.020% THC by weight. Generally, a low content of THC or THCV and/or other cannabinoids, in particular psychoactive cannabinoids is desirable.

As disclosed above, the composition may, or may not comprise further cannabinoids, in some embodiments, such further cannabionoid(s) is/are psychoactive cannabionoid(s), such as THC and/or THCV; and/or cannabinoid(s) binding to a CB1 receptor. In some embodiments, the further cannabionoid(s) is/are non-psychoactive cannabionoids, such as one or more cannabinoid(s) selected from: THCA, CBDA, CBG, CBC, CBL, CBV, THCP, CBDV, CBCV, CBGV, CBGM, CBE, and CBT; and/or cannabinoid(s) not binding to a CB1 receptor. In some embodiments, the further cannabionoid(s) is/are selected from or one or more of THC, THCA, CBDA, CBG, CBC, CBL, CBV, THCV, THCP, CBDV, CBCV, CBGV, CBGM, CBE, and CBT, including any combination(s) thereof.

Conventional nasal sprays comprise salt, such as physiological saline solution, and/or around 0.9% NaCl. In contrast, in some embodiments, the composition does not comprise sodium chloride (saline), and/or comprises less than 0.1 or 0.05% (w/w) NaCl.

Likewise, conventional nasal sprays comprise water. In contrast, in some embodiments, the composition does not comprise water, such as added water, and/or less than 1.0, 0.5, or 0.1% (w/w) water.

Surprisingly and unexpectedly, a nasal spray as disclosed herein without NaCl and/or water provides not only good results, but it is also pleasant to use.

In some embodiments, the hemp oil comprises ≤0.2% or ≤0.05% CBDV by weight; ≤0.2% or ≤0.05% CBDA by weight; ≤0.5% or ≤0.025% CBG by weight; and ≤0.05% or ≤0.020% THC by weight.

In some embodiments, the lavender oil possesses CAS no. 8000-28-0 and INCI name: Lavandula angustifolia OIL. In some embodiments, the lavender oil comprises (by weight): ˜0.05% coumarin, CAS No. 91-64-5; ˜0.40% geraniol, CAS No. 106-21-1; ˜0.5% D-limonene, CAS no. 5989-27-5; ˜30% Linalool, CAS no. 78-70-6; and a VOC-CH content of ˜1%. In some embodiments, the lavender oil comprises one or more of: ˜0.05% coumarin, CAS No. 91-64-5; ˜0.40% geraniol, CAS No. 106-21-1; ˜0.5% D-limonene, CAS no. 5989-27-5; and/or ˜30% Linalool, CAS no. 78-70-6. A suitable lavender oil can e.g. be provided from www.voegele-ingredients.de.

In some embodiments, the sesame oil is a refined oil with the following specifications: acid ≤0.5, peroxide value ≤10.0, unsaponified matter ≤2% (w/w), alkaline substances ≤0.1, water ≤0.1%. Concerning the triglyceride composition, in some embodiments a refined sesame oil comprises (by weight): LLL 7-19% LLL, 13-30% OLL, 5-9% PLL, 12-23% OOL, 6-14% POL, 5-16% OOO, 2-8% SOL, and 2-10% POO. The fatty acid radicals are designated as linoleic (L), oleic (O), palmitic (P), and stearic (S). The abbreviations for triglycerides used are: trilinolein (LLL), 1,2-dilinoleoyl-3-oleoyl-rac-glycerol (OLL), 1,2-dilinoleoyl-3-palmitoyl-rac-glycerol (PLL), 1,2-dioleoyl-3-linoleoyl-rac-glycerol (OOL), 1-palmitoyl-2-oleoyl-3-linoleoyl-rac-glycerol (POL), triolein (OOO), 1-linoleoyl-2-oleoyl-3-stearoyl-rac-glycerol (SOL), and 1,2-dioleoyl-3-palmitoyl-rac-glycerol (POO). Suitable sesame oils are e.g. available from www.oelmuehle-hartmann.de.

In some embodiments, the hemp-, lavender- and sesame oil are as specified above. The use of such compositions is believed to provide a product with satisfying properties in the context of the present invention, i.e. when provided in appropriate amounts, such as specified herein.

In some embodiments, suitable compositions may also be characterized by one or more, or all of the following features: (a) the concentration of CBD is lower than CBN (e.g. a ratio around 1:2); (b) concentration of CBD dissolved in hemp oil 1-10% or 2-5% *; (c) concentration of CBN dissolved in hemp oil: 2-20%, or 4-10%; (d) absence of other cannabinoids, in particular THC or other psychoactive components in physiologically active amounts; (d) absence of saline and/or alcohol.

In some embodiments, CBD and/or CBN can be dissolved in one or more of sesame-, hemp-, and lavender oil, thus not only in hemp oil.

Generally, non-hallucinogenic cannabinoids are preferred in order to avoid undesired side-effects upon use or treatment with composition(s) comprising such compounds, in particular when they are present in physiologically active amounts.

Further suitable concentrations and/or concentration ranges may be disclosed herein.

Concerning the CBD used in the preparation or formulation of a CBD-comprising composition, in some embodiments, the CBD used in the provision of the composition is crystalline. In some embodiments, the CBD is “type A CBD”. Often, the use and/or presence of “type A CBD” is preferred in contrast to “type B CBD”.

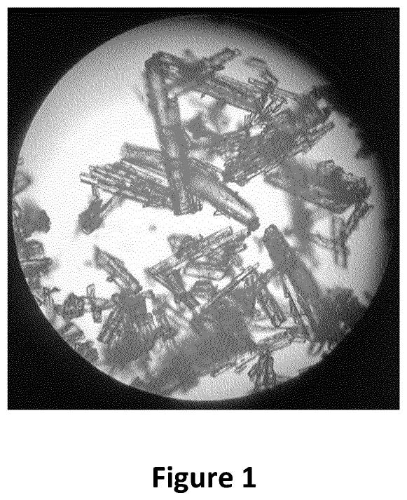

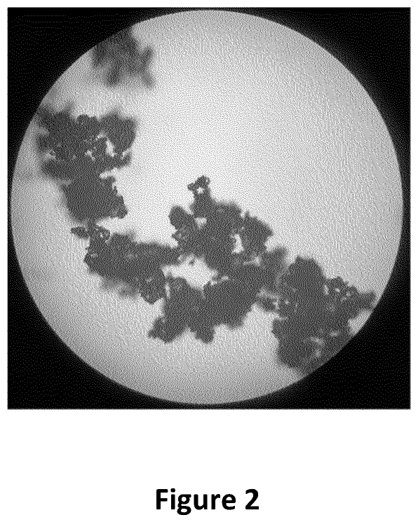

In some embodiments, the CBD used for providing a composition as disclosed above is characterized by certain features, such as the crystal structure, type and/or conformation. It has been observed by the inventors, see e.g. Example 10, that CBD with a needle-like crystal structure (=crystal structure A; see FIG. 1), surprisingly and unexpectedly, appears significantly more potent than CBD with a different crystal structure, a non-needle like structure, also termed “bunch-like or “cluster-like” herein (=crystal structure B; see FIG. 2).

In some embodiments, the CBD possesses, when crystalline, or is capable of forming a needle-like crystal structure. In some embodiments, CBD of crystal structure A (or capable of forming needle-like crystals) is at least 1.5, 2, 3, 4, 5, 7.5, 10, 15 or 20 times more potent on a weight/weight basis than CBD of crystal structure B (or capable of forming cluster/bunch-like crystals).

CBD of crystal structure A, or CBD capable of forming needle-like crystals, is also called “type A CBD” herein, while CBD of crystal structure B, or CBD capable of forming “bunch-like or “cluster-like” crystals is called “type B CBD”. In some embodiments, the CBD is “type A CBD”. Often, “type A CBD” is preferred in contrast to “type B CBD”.

It can be speculated, if the CBD needs to be in an active form, such one or more specific conformation(s) in order to be active upon administration to a subject, such as in a topical formulation. Lack of activity or potency can also be caused by a lower uptake rate and/or difficulties in passing through the skin.

Without wanting to be bound by any theory, it is believed that the difference in crystal structure may be caused by a different molecular structure, such as a different conformation. This could e.g. be due to a failure of the subject's body to recognize the “wrong” CBD conformation or the like. It is conceivable that the differences in CBD crystal structure are caused by a different extraction process. In particular, the CBD disclosed in FIG. 1 was provided by an extraction process, comprising extraction with isopropanol, distillation and crystallization with heptane (see e.g. Example 9), while the CBD disclosed in FIG. 2 was provided by critical CO₂ extraction.

Generally, crystalline CBD can be provided by methods and techniques known in the art, such as by methods disclosed in U.S. Pat. No. 10,413,845 and/or U.S. Pat. No. 10,414,709.

In short, crystalline CBD can be provided from hemp or Cannabis (Cannabis sativa) by a method consisting essentially of:
Extracting hemp or Cannabis with e.g. isopropanol to produce an extract rich in cannabinoids, THC, CBD and terpenes
Evaporating the solvent portion of the extract to generate a substantially solvent-free extract
Distilling the substantially solvent-free extract to isolate the CBD, and
Crystallizing the distilled, isolated CBD to produce a crystallized, isolated CBD and one or more recrystallization(s) if needed by the use of a suitable organic solvent, such as an alkane, e.g. heptane, commonly followed by
Solvent removal by e.g. vacuum drying. to remove volatile remnants.

Thus, in some embodiments, the CBD crystals used in the formulation and/or provision of the topical composition are needle-like crystals, such as crystals shown in FIG. 1. Likewise, in some embodiments, the CBD crystals used in the formulation of the topical composition are not cluster- or bunch-shaped, such as crystals similar to crystals shown in FIG. 2.

In some embodiments, the CBD crystals used in the provision and/or formulation of the topical composition are not provided by an extraction method comprising critical CO₂ extraction.

In some embodiments, the CBD crystals used in the provision and/or formulation of the topical composition are provided by a method comprising extraction with a C₃-C₄ alcohol, such as isopropanol, and one or more crystallisations steps with a C₆-C₈ alkane, such as heptane. In some embodiments, the C₃-C₄ alcohol is isopropanol. In some embodiments, the C₆-C₈ alkane is heptane. In some embodiments, the C₃-C₄ alcohol is isopropanol, and the C₆-C₈ alkane is heptane. This combination is believed to provide CBD crystals of satisfactory quality, such as absence or reduction in inhibitors and/or the desired conformation of the CBD.

In some embodiments, a suitable CBD composition or product can be obtained when the CBD crystals are provided by a method comprising CO₂ extraction, in particular critical CO₂ extraction and one or more crystallisations steps with a C₆-C₈ alkane, such as heptane.

As seen in Table 1, it can be seen that the Cannabinoid profile of type A and type B CBD can be rather similar.

TABLE 1
Analysis of CBD of crystal structure A versus crystal structure B

| Cannabinoid profile | Type A | Type B |
|---|---|---|
| CBD | 99.33% | 98.60% |
| CBDV | 0.39% | 0.19% |
| CBDA | 0.01% | n.d. |
| CBG | n.d. | n.d. |
| CBN | 0.04% | n.d. |
| THC | n.d. | n.d. |

n.d. not detected; type A CBD was sourced from Enecta, type B CBD was sourced from Pharma Hemp

It is, however, also conceivable that the differences in crystal structure, can relate to and be caused by different extraction processes. Different crystal structures can also be indicative of different concentrations of “CBD inhibitors”, and/or different concentrations of “CBD enhancers”. In some embodiments, terpenes, such as naturally occurring terpenes, in particular terpenes found in plants, such as in Cannabis sativa, act as CBD inhibitors, which is not desirable.

Thus, in some embodiments, CBD of crystal structure B alias “type B CBD” can be converted to CBD of crystal structure A alias “type A CBD” (and/or CBD capable of forming crystal structure A) by an organic extraction step and/or recrystallisation step. In such embodiments, it is conceivable that the change in crystal structure is related to the presence of inhibitors that are reduced significantly in the additional extraction and/or crystallization step(s). Alternatively, the organic extraction step may provide a change in conformation of the CBD, rendering it more active again. In some embodiments, recrystallization with heptane can change the B-type CBD into A-type CBD.

In some embodiments, CBD of crystal structure B has been provided by critical CO₂ extraction, such as CBD crystals provided by www.pharma-hemp.com and/or following a similar extraction protocol as said manufacturer.

In some embodiments, presence of terpenes and/or terpenoids, in particular Cannabis sativa terpenes or in a CBD-comprising topical composition as disclosed herein, provides one or more undesirable effect(s), such as one or more of: reduced efficiency or potency, inability or reduced ability to recognize the CBD, need for a higher CBD formulation for obtaining similar effect, increase in non-CBD cannabinoids in the formulation. In some embodiments, said composition comprises 0.0001% or less, 0.001% or less, 0.01% or less, or 0.1% or less terpenes, in particular Cannabis sativa terpenes, by weight.

In some embodiments, the crystalline CBD does not comprise significant amounts of terpenes, such as less than 0.1, less than 0.05, less than 0.02, less than 0.01, less than 0.005, less than 0.002, less than 0.001% terpenes by weight.

It is also conceivable that other plant components, such as terpenoids can act as inhibitors. In some embodiments the presence of terpenoids, such as Cannabis sativa terpenoids can be undesirable. In some embodiments, the crystalline CBD does not comprise significant amounts of terpenoids, such as less than 0.1, less than 0.05, less than 0.02, less than 0.01, less than 0.005, less than 0.002, less than 0.001% terpenoids by weight.

In some embodiments, the use of CBD having or capable of providing crystals of crystal structure A, such as shown in FIG. 1 in a CBD-comprising composition as disclosed herein, provides a positive effect, such as one or more of: increased efficiency, possibility to reduce total amount of CBD in the formulation, the subject needs less intranasal formulation to achieve the same effect, improved recognition and/or CBD uptake by the subject's body, reduction in non-CBD cannabinoids in the formulation and/or other impurities.

Generally, compositions according to the first aspect can be provided using methods and procedures known in the art. In some embodiments, compositions according to the first aspect can be provided as shown in the second aspect and/or Examples.

In some embodiments, the CBD is “type A CBD”. Often, the use of “type A CBD” is preferred in contrast to “type B CBD”.

Further embodiments concerning different compositions according to the present invention are also disclosed in the Examples.

In a second aspect, the present invention relates to a method for providing a composition for nasal application according to the first aspect. Such a method may comprise the steps or acts of (i) providing hemp oil comprising CBD and/or CBN; (ii) providing lavender oil, sesame oil and vitamin E (e.g. vitamin E oil); and (iii) mixing the hemp oil from step (i) with the ingredients from step (ii); and optionally (iv) aliquoting the composition of step (iii) into one or more receptacle(s), such as nasal pump spray bottle(s) adapted to provide 50-350 μl, 100-250 μl, or around 160 μl per puff.

In some embodiments, the CBD is “type A CBD”. Often, the use of “type A CBD” is preferred in contrast to “type B CBD”.

In some embodiments, a method is disclosed for providing a composition for nasal application according to any one of the preceding claims, comprising the steps or acts of:
i. Providing hemp oil comprising CBD and/or CBN;
ii. Providing lavender oil, sesame oil and vitamin E (e.g. vitamin E oil); and
iii. Mixing the hemp oil from step (i) with the ingredients from step (ii); and optionally
iv. Aliquoting the composition of step (iii) into one or more receptacle(s), such as nasal pump spray bottle(s) adapted to provide 50-350 μl, 100-250 μl, or around 160 μl per puff.

In some embodiments, the aliquot size is comparable to conventional nasal sprays on the market. In some embodiments, the aliquot size is 1-20, 2-15, 3-12, or 5-10 ml.

Alternatively, CBD and/or CBN can be dissolved in one or more of hemp oil, sesame oil, and/or lavender oil. In some embodiments, CBD and/or CBN are dissolved in sesame oil, or an oil mixture comprising hemp oil and/or sesame oil, and optionally lavender oil.

Generally, it is believed that protecting the composition from electromagnetic radiation, such as UV or visible light increase storability, such as when storing at room temperature. This can be provided by means known in the art, such as light-tight packaging. In some embodiments, the receptacle provides protection from UV- and/or visible light.

In a third aspect, the present invention pertains to a composition, such as a composition for intra-nasal application, provided by a method according to the second aspect.

In a fourth aspect, the present invention concerns a receptacle comprising a composition, such as a composition for intra-nasal application, according to the first, third, seventh or eighth aspect. In some embodiments, such as receptacle can be a nasal pump spray bottle, such as or similar to nasal spray bottles commonly sold, e.g. “Nasivin”, comprising oxymetazoline hydrochloride, e.g. 10 ml or the like.

In some embodiments, the receptacle provides light- and/or UV-protection to the composition.

In some embodiments, the receptacle is a nasal pump spray bottle, such as a pump bottle for administering 50-350 μl, 100-250 μl, or around 160 μl per puff per nostril.

In some embodiments, the receptacle is adapted to accommodate a volume of 1-20, 2-15, 3-12, or 5-10 ml of the composition for nasal application.

In a fifth aspect, the present invention relates to a kit comprising a receptacle according to the fourth aspect, and optionally, comprising an instruction for use.

In some embodiments, the kit comprises a packaging, such as carton or the like for said receptacle and/or instruction for use.

In order to provide protection from light and/or UV, in some embodiments, the packaging may provide light and/or UV protection. Thus in some embodiments, the receptacle and/or the packaging may provide protection from UV and/or visible light.

In a sixth aspect, the present invention pertains to a method for treatment of a subject, comprising intranasal application or administration of a composition as disclosed herein, such as a composition according to the first, third, seventh or eighth aspect.

In some embodiments, administration of said composition can be performed as follow these instructions for use: The nasal spray delivers the target dose (e.g. 160 μl) per spray (or “puff”), which is operated manually to deliver the content by pressing the plunger base towards the flange until it stops. Using a nasal spray: (1) close the nostril that is not receiving the medication. Do this by gently pressing on that side of your nose. (2) Gently insert the bottle tip into the other nostril. (3) Breathe in deeply through that nostril as you squeeze the bottle (pressing the plunger base towards the flange until it stops) and apply one spray intranasal. (4) Repeat steps 1-4 for the other nostril. In some embodiments, more than 1 puff per nostril can be needed, if larger doses are required. Usually, administration of the nasal composition is performed using both nostrils. Alternatively, the application can also be performed using one nostril only.

In some embodiments, the subject is an animal or a human.

In some embodiments, the subject is an infant, child, adolescent, adult or senior.

In some embodiments, the dosage regimen is 50-350 μl, 100-250 μl, or around 160 μl in each nostril. Said application is usually performed in both nostrils. Alternatively, the application can also be performed using one nostril only. It can then be advisable, to double the dosage/volume.

In some embodiments, the dosage regimen is a total of 0.5-5, 1.5-3.5, or around 2.8 mg CBN per application usually comprising both nostrils. Said application is usually performed in both nostrils. Alternatively, the application can also be performed using one nostril only.

In some embodiments, the dosage regimen is a total of 0.5-4, 0.8-1.8, or around 1.4 mg CBD per application usually comprising both nostrils. Said application is usually performed in both nostrils. Alternatively, the application can also be performed using one nostril only.

In some embodiments, the dosage regimen is a total of0.5-5, 1.5-3.5, or around 2.8 mg CBN and a total of 0.5-4, 0.8-1.8, or around 1.4 mg CBD per application usually comprising both nostrils. Said application is usually performed in both nostrils. Alternatively, the application can also be performed using one nostril only.

In some embodiments, the subject is suffering from a sleep disorder related to: Insomnia, Snoring, Obstructive Sleep Apnoea, Sleep Hypoventilation, Restless Legs Syndrome, Bruxism, Narcolepsy, Sleep talking, sleep walking and/or other automatic behaviour(s), Nightmares and/or night terrors, Rapid eye movement behaviour disorder.

In some embodiments, treatment may also concern conditions like jet lag, anxiety, and/or difficulties in falling asleep under unusual circumstances, e.g. when travelling, e.g. not sleeping at home, e.g. during transport in a bus, car, train, aeroplane, hotel, pension, boarding school, prison, or in a hospital, hospice, or the like, as well as during military service or similar services. In some embodiments, the present composition(s) provide a positive effect and/or treatment in a condition related to Parkinson, multiple sclerosis (MS), muscle spasmed, anxiety, depression, Alzheimer, epilepsy, pain, pain relief, and/or neurological conditions requiring a neuroprotective effect.

Concerning the effect of a treatment according to the present invention, this may comprise, in particular but not exclusively relating to sleeping disorders and/or anxiety, such an effect may comprise one or more of:
Positive effect within 10-15 minutes after administration
Increase in coherent sleep
Reduce in number of waking-up during the intended sleeping period
Increased feeling of freshness the day after
The subject does not fee drugged
Decrease the drowsiness
Increase the deep sleep
Increase the quality of life
Does not make the person groggy
Application of one spray in each nostril is enough 10-15 minutes before bedtime
Makes the muscles relax
Calms the body and mind
Less nightmares,
including any combination(s) thereof.

Further desirable effects may comprise one or more measurable pattern(s) or behaviour(s) detectable by means common in the field, such as by polysomnography and/or actigraphy (see e.g. Example 8).

In a seventh aspect, the present invention concerns a composition according to the first, third, or eighth aspect for use as a medicament and/or therapeutic agent. This may comprise treatment of one or more sleep disorder(s), such as and/or related to one or more of: Insomnia; Snoring; Obstructive Sleep Apnoea; Sleep Hypoventilation; Restless Legs Syndrome; Bruxism; Narcolepsy; Sleep talking, sleep walking and/or other automatic behaviours; Nightmares and/or night terrors; and/or Rapid eye movement behaviour disorder. In some embodiments, this may also concern conditions and/or effects as disclosed herein, e.g. according to the sixth aspect.

In an eighth aspect, the present invention pertains to a pharmaceutical composition comprising or consisting essentially of a composition according to the first, third or seventh aspect, optionally comprising one or more pharmaceutically acceptable carrier(s) and/or diluent(s).

In a ninth aspect, the present invention concerns a CBD-comprising composition, such as an intranasal composition and/or composition for intra-nasal application, wherein the CBD used in the formulation is crystalline and/or “type A CBD”. In some embodiments, said composition is a composition as disclosed in the first, third, seventh or eighth aspect. In some embodiments, the CBD is of type A (e.g. needle-like crystals) and/or capable of forming needle-like crystals. as disclosed herein, e.g. in the first aspect and/or Examples.

In a tenth aspect, the present invention pertains to a dosage regimen, comprising administering a composition, such as a CBD- and/or CBD comprising composition such as an intra-nasal composition as disclosed herein, e.g. according to the first, third, seventh, eighth or ninth aspect. In some embodiments, the CBD is of “type A” A (e.g. needle-like crystals) and/or capable of forming needle-like crystals. as disclosed herein, e.g. in the first aspect and/or Examples.

The current invention is further exemplified in the following section. These examples are, however, not to be construed as limiting to the present invention.

### EXAMPLES

### Example 1—Provision of Nasal Sleep Compositions

Generally, compositions can be formulated using methods and equipment customary in the field.

Unless indicated otherwise, percentages are % by weight.

Generally, crystalline CBD is sourced from Enecta, unless indicated otherwise Hemp oil comprising CBD and/or CBN is provided, e.g. by adding a suitable quantity of CBD and/or CBN to hemp oil. Lavender oil, sesame oil and vitamin E (e.g. vitamin E oil) are provided in desired amounts (by weight) and mixing the CBD and/or CBN comprising hemp oil. The compositions are aliquoted into suitable receptacles, such as nasal pump spray bottles, and kept protected from light. Preferably, the compositions are stored in UV- and light-tight receptacles. For long term storage, compositions are stored at 10-25 degrees Celsius.

Concerning the CBD- and CBN comprising hemp oil used for composition A, the certificate of analysis comprises the following details: Product: 2.5% CBD 5% CBN Hemp Oil, Analysis N° 20072002, Product lot N° Q0720L-0; Yellow-green Oil; Analysis Date Jul. 23, 2020;

Test results and prescribed limits:

Assay (HPLC) CBD 2.48% 2.5±0.50%; CBN 5.07% 5.0±0.50%;

Related substances (%): CBDV 0.03%≤0.20%; CBDA 0.02%≤0.20%; CBG 0.01%≤0.50%; THC 0.01%≤0.05%;

Further analysis: KF 0.1%≤0.5%; Colour (420 nm) 0.108 AU≤0.300 AU; Total ashes 0.08%≤0.30%; Density 0.932 g/ml≤0.950 g/ml; Viscosity 52 mPa≤150 mPa; Peroxides 9 meq O₂/kg≤15 meq O₂/kg;

Heavy metals: Arsenic ongoing ppm≤1.5 ppm; Cadmium ongoing ppm≤0.5 ppm; Mercury ongoing ppm≤3.0 ppm; Lead ongoing ppm≤0.5 ppm

Microbiology: Total bacterial count 35 cfu/g≤1000 cfu/g; Yeasts and Moulds 30 cfu/g≤100 cfu/g; Salmonella sp: Absent/25 g; E. coli: Absent/10 g; P. aeruginosa: Absent/1 g; Staphylococci coagulase positive: Absent/1 g

For other CBD and/or CBN concentrations, these can e.g. be provided by dissolving appropriate CBD and/or CBN quantities in a suitable oil, such as hemp oil. CBD and CBD are provided as “crystalline” or “pure” CBD in powder-form, with a purity of at least 98%, and comprising less than 1.5% (w/w) of CBDV, CBG, and/or CBN, and less than 0.05% THC.

### Formulation A—“CBN+CBD” (0.42% CBD and 0.85% CBN):

| | Purity* | Weight | Concentration |
|---|---|---|---|
| Ingredient | (%) | (g) | % (w/w) |
| Hemp oil (comprising 2.5% | 92.5 | 12.5 | 16.98 |
| CBD and 5% CBN by | | | |
| weight) | | | |
| Lavender oil - russisch, | 100 | 0.02 | 0.03 |
| Vögele, 00002368 EP grade | | | |
| Sesam Oil (Ölmühle | 100 | 60.1 | 81.64 |
| Hartman) EP grade | | | |
| Vitamin E - Oil - EP | 40 | 1 | 1.36 |
| grade** | | | |
| | Total | 73.62 | 100.00 |

*% active compound in ingredient
**0.4 g Tocopherol equivalent to 1 g Vit. E oil

### Formulation B—“CBD, No CBN” (0.42% CBD):

Formulation comprises the use of a hemp oil similar to the one used in formulation A, however not comprising any CBN in significant amounts.

| | Purity* | Weight | Concentration |
|---|---|---|---|
| Ingredient | (%) | (g) | % (w/w) |
| Hemp oil (comprising 2.5% | 92.5 | 12.5 | 16.98 |
| CBD) | | | |
| Lavender oil - russisch, | 100 | 0.02 | 0.03 |
| Vögele, 00002368 EP grade | | | |
| Sesam Oil (Ölmühle | 100 | 60.1 | 81.64 |
| Hartman) EP grade | | | |
| Vitamin E - Oil - EP | 40 | 1 | 1.36 |
| grade** | | | |
| | Total | 73.62 | 100.00 |

*% active compound in ingredient
**0.4 g Tocopherol equivalent to 1 g Vit. E oil

### Formulation C—Placebo

| | Purity* | Weight | Concentration |
|---|---|---|---|
| Ingredient | (%) | (g) | % (w/w) |
| Sesam Oil (Ölmühle | 100 | 60.1 | 98.64 |
| Hartman) EP grade | | | |
| Vitamin E - Oil - EP | 40 | 1 | 1.36 |
| grade** | | | |
| | Total | 61.1 | 100.00 |

*% active compound in ingredient
**0.4 g Tocopherol equivalent to 1 g Vit. E oil

### Example 2—Application/Use of Nasal Sleep Compositions

Different formulation for intra-nasal administration by “nose spray” are administered according to the instructions provided to the test subjects:

The nasal spray delivers the target dose of 160 μl per spray, which is operated manually to deliver the content by pressing the plunger base towards the flange until it stops.

Using a nasal spray:
1. Close the nostril that is not receiving the medication. Do this by gently pressing on that side of your nose.
2. Gently insert the bottle tip into the other nostril.
3. Breathe in deeply through that nostril as you squeeze the bottle (pressing the plunger base towards the flange until it stops) and apply one spray intranasal.
4. Repeat steps 1-4 for the other nostril.

Multiple doses can be provided by repeating step 3 if needed.

### Example 3—Inclusion & Exclusion Criteria for Sleep Study:

INCLUSION CRITERIA:
18 to 60-year-old men and women
Generally healthy with below mild to moderate form of one of the following sleep disorders (SD): (1) Insomnia, (2) Snoring, (3) Obstructive Sleep Apnea, (4) Sleep Hypoventilation, (5) Restless Legs Syndrome, (6) Bruxism, (7) Narcolepsy, (8) Sleep talking, sleep walking and other automatic behaviours, (9) Nightmares and night terrors, and (10) Rapid eye movement behaviour disorder.
Subjects are for more than 1 month's sleep-deprived, defined as sleeping on a regular basis less than or equal to approximately 6,5 hours/night by history and/or objective devices (wrist activity monitors and sleep logs).

INCLUSION CRITERIA: External comparison subjects for extension of Effectiveness Study must meet the criteria above.

EXCLUSION CRITERIA: Diagnosed sleep disorders including: (a) Chronic insomnia; (b) Untreated sleep disordered breathing (sleep apnoea at a level of severity [using standardized criteria for measurement], or diagnosed UARS [upper airway resistance syndrome] that would impair the ability to increase sleep duration [Intervention Group] or maintain sleep duration [Comparison Group]; (c) Central apnea; (d) Unstable weight (voluntary losses in BMI greater than 5% over the past 6 months); currently being enrolled in a weight loss program; (c) Untreated or uncontrolled diabetes; (f) Severe uncontrolled hypertension; (f) Other chronic organ disease diagnosis including: COPD, Chronic cardiac arrhythmia requiring treatments, and Gastro-esophageal disorders associated with sleep-related symptoms (g) Medications: chronic use of prescription or over-the-counter medications known to affect sleep (e.g., systemic steroids, NSAIDs); current anticonvulsant therapy; (h) Chronic fatigue syndrome and fibromyalgia; (i) Acromegaly, hypothyroidism (unless on a stable replacement dose of thyroid hormone), Cushing disease or other endocrine disorders known to affect sleep; (j) Poorly controlled major depression (subjects who have been on a stable pharmacological antidepressant treatment for 3 months and are in remission without substantial weight gain are eligible); (k) Other current DSM-IV diagnoses, including: Eating disorders such as bulimia nervosa and binge eating disorder; Anxiety disorders such as PTSD and panic attacks; Mania; and Schizophrenia; (1) Medication and substance abuse such as excessive alcohol consumption or drug abuse or dependence that may pose a threat to compliance; (m) Being a rotating worker, shift worker (working evenings or nights), or long distance commuter (more than approximately 90 minutes each way), traveling frequently outside of time zone; being in an occupation that may require special vigilance such as driving a truck, bus, or cab; operating heavy machinery; being a pilot or air traffic controller; (n) Being likely to move to a different geographical area during the study; (o) Having a sleep partner that would make compliance with study requirements difficult: (p) Pregnancy and lactation; (o) Menopause: (q) Chronic excessive caffeine use (habitual intake of more than 500 mg/day).

### Example 4—Test Results (Formulations A-C)

### Formulation A “CBN+CBD”

| Test person | 1 | 2 | 3 | 4 | 5 | 6 |
|---|---|---|---|---|---|---|
| Male (M) or Female (F) | M | M | F | M | F | F |
| SD type (see Example 3, incl. criteria) | SD: 1 | SD: 1 | SD: 5 | SD: 1 | SD: 1 | SD: 1 |
| Test period(days) | 8 | 7 | 7 | 9 | 8 | 7 |
| How many puffs (spray's per nostril) did you | 1 | 2 | 1 | 1 | 1 | 1 |
| apply before going to bed? | | | | | | |
| How much do the sleep disorder effect your | 3 | 4 | 5 | 4 | 5 | 5 |
| life - scaled on a 0-10 scale (0 = no effect; | | | | | | |
| 10 = worst ever experienced - always sleepy) | | | | | | |
| before testing Sleep Nasal Spray? | | | | | | |
| How fast did you fall asleep in average before | B: 15 | B: 90 | B: 90 | B: 120 | B: 100 | B: 90 |
| testing Sleep Nasal spray (in minutes)? | | | | | | |
| How fast did you fall asleep in average during | D: 2 | D: 15 | D: 15 | D: 15 | D: 10 | D: 15 |
| test period of Sleep nasal spray (in minutes)? | | | | | | |
| How much coherent sleep, did you get in | B: 3 | B: 2 | B: 2.5 | B: 2 | B: 2 | B: 2 |
| average before testing Sleep Nasal spray | | | | | | |
| (in hours)? | | | | | | |
| How much coherent sleep, did you get in | D: 7 | D: 6 | D: 7 | D: 7 | D: 6 | D: 7 |
| average During test period of Sleep nasal | | | | | | |
| spray (in hours)? | | | | | | |
| How many times did you wake up during the | B: 3 | B: 4 | B: 4 | B: 3 | B: 4 | B: 3 |
| night before testing Sleep Nasal spray (in | | | | | | |
| hours)? | | | | | | |
| How many times did you wake up during the | D: 0 | D: 1 | D: 1 | D: 1 | D: 1 | D: 0 |
| night During testing Sleep Nasal spray (in | | | | | | |
| hours)? | | | | | | |
| How fresh/awake did you feel the day after - | 6 | 3 | 2 | 3 | 2 | 2 |
| before testing the Sleep Nasal spray- scaled | | | | | | |
| on a 0-10 scale? 0: Not fresh at all - very | | | | | | |
| sleepy-10: Very fresh and ready for the day | | | | | | |
| How fresh/awake did you feel the day after | 9 | 8 | 8 | 8 | 9 | 8 |
| using the Sleep Nasal spray- scaled on a 0-10 | | | | | | |
| scale? 0: Not fresh at all - very sleepy-10: | | | | | | |
| Very fresh and ready for the day | | | | | | |
| How deep a sleep did you feel you have had | 2 | 2 | 2 | 2 | 3 | 2 |
| during the night before testing the Sleep | | | | | | |
| Nasal spray- scaled on a 0-10 scale? 0: No | | | | | | |
| deep sleep-10: Very good and deep a sleep | | | | | | |
| How deep a sleep did you feel you have had | 8 | 8 | 8 | 8 | 7 | 8 |
| during the night during testing the Sleep | | | | | | |
| Nasal spray- scaled on a 0-10 scale? 0: No | | | | | | |
| difference than before not using Sleep Nasal | | | | | | |
| spray-10: Very good and deep a sleep | | | | | | |
| Medication reduced during test period? | NA | NA | NA | NA | NA | NA |

### Formulation B “CBD”

| Test person | 1 | 2 | 3 | 4 | 5 | 6 |
|---|---|---|---|---|---|---|
| Male (M) or Female (F) | F | F | M | M | F | F |
| SD type (see Example 3, incl. criteria) | SD: 1 | SD: 1 | SD: 1 | SD: 1 | SD: 5 | SD: 1 |
| Test period(days) | 7 | 6 | 7 | 8 | 7 | 7 |
| How many puffs (spray's per nostril) did you | 1 | 1 | 1 | 2 | 1 | 1 |
| apply before going to bed? | | | | | | |
| How much do the sleep disorder effect your | 4 | 5 | 5 | 4 | 4 | 5 |
| life - scaled on a 0-10 scale (0 = no effect; | | | | | | |
| 10 = worst ever experienced - always sleepy) | | | | | | |
| before testing Sleep Nasal Spray? | | | | | | |
| How fast did you fall asleep in average before | B: 90 | B: 60 | B: 90 | B: 60 | B: 90 | B: 90 |
| testing Sleep Nasal spray (in minutes)? | | | | | | |
| How fast did you fall asleep in average during | D: 15 | D: 30 | D: 20 | D: 15 | D: 30 | D: 20 |
| test period of Sleep nasal spray (in minutes)? | | | | | | |
| How much coherent sleep, did you get in | B: 2.5 | B: 2 | B: 2 | B: 3 | B: 3 | B: 2 |
| average before testing Sleep Nasal spray | | | | | | |
| (in hours)? | | | | | | |
| How much coherent sleep, did you get in | D: 2.5 | D: 3 | D: 4 | D: 5 | D: 5 | D: 5 |
| average during test period of Sleep nasal | | | | | | |
| spray (in hours)? | | | | | | |
| How many times did you wake up during the | B: 4 | B: 3 | B: 3 | B: 4 | B: 3 | B: 4 |
| night before testing Sleep Nasal spray (in | | | | | | |
| hours)? | | | | | | |
| How many times did you wake up during the | D: 2 | D: 2 | D: 1 | D: 1 | D: 2 | D: 2 |
| night during testing Sleep Nasal spray (in | | | | | | |
| hours)? | | | | | | |
| How fresh/awake did you feel the day after - | 6 | 4 | 3 | 3 | 3 | 2 |
| before testing the Sleep Nasal spray- scaled | | | | | | |
| on a 0-10 scale? 0: Not fresh at all - very | | | | | | |
| sleepy-10: Very fresh and ready for the day | | | | | | |
| How fresh/awake did you feel the day after | 4 | 5 | 6 | 6 | 5 | 6 |
| using the Sleep Nasal spray- scaled on a 0-10 | | | | | | |
| scale? 0: Not fresh at all - very sleepy-10: | | | | | | |
| Very fresh and ready for the day | | | | | | |
| How deep a sleep did you feel you have had | 2 | 2 | 3 | 2 | 2 | 3 |
| during the night before testing the Sleep | | | | | | |
| Nasal spray- scaled on a 0-10 scale? 0: No | | | | | | |
| deep sleep-10: Very good and deep a sleep | | | | | | |
| How deep a sleep did you feel you have had | 4 | 3 | 5 | 6 | 5 | 7 |
| during the night during testing the Sleep | | | | | | |
| Nasal spray- scaled on a 0-10 scale? 0: No | | | | | | |
| difference than before not using Sleep Nasal | | | | | | |
| spray-10: Very good and deep a sleep | | | | | | |
| Medication reduced during test period? | NA | NA | NA | NA | NA | NA |

### Formulation C—“Placebo”

| Test person | 1 | 2 | 3 | 4 | 5 | 6 |
|---|---|---|---|---|---|---|
| Male (M) or Female (F) | M | M | M | F | F | F |
| SD type (see Example 3, incl. criteria) | SD: 1 | SD: 1 | SD: 1 | SD: 1 | SD: 1 | SD: 5 |
| Test period(days) | 7 | 6 | 7 | 6 | 7 | 7 |
| How many puffs (spray's per nostril) did you | 2 | 1 | 1 | 2 | 1 | 1 |
| apply before going to bed? | | | | | | |
| How much do the sleep disorder effect your | 3 | 4 | 5 | 4 | 5 | 4 |
| life - scaled on a 0-10 scale (0 = no effect; | | | | | | |
| 10 = worst ever experienced - always sleepy) | | | | | | |
| before testing Sleep Nasal Spray? | | | | | | |
| How fast did you fall asleep in average before | B: 60 | B: 60 | B: 90 | B: 60 | B: 60 | B: 60 |
| testing Sleep Nasal spray (in minutes)? | | | | | | |
| How fast did you fall asleep in average during | D: 40 | D: 50 | D: 60 | D: 60 | D: 60 | D: 60 |
| test period of Sleep nasal spray (in minutes)? | | | | | | |
| How much coherent sleep, did you get in | B: 3 | B: 3 | B: 2 | B: 3 | B: 2 | B: 3 |
| average before testing Sleep Nasal spray | | | | | | |
| (in hours)? | | | | | | |
| How much coherent sleep, did you get in | D: 3 | D: 4 | D: 3 | D: 3 | D: 2 | D: 3 |
| average during test period of Sleep nasal | | | | | | |
| spray (in hours)? | | | | | | |
| How many times did you wake up during the | B: 3 | B: 3 | B: 4 | B: 2 | B: 3 | B: 3 |
| night before testing Sleep Nasal spray (in | | | | | | |
| hours)? | | | | | | |
| How many times did you wake up during the | D: 3 | D: 2 | D: 4 | D: 2 | D: 3 | D: 2 |
| night during testing Sleep Nasal spray (in | | | | | | |
| hours)? | | | | | | |
| How fresh/awake did you feel the day after - | 5 | 3 | 2 | 4 | 3 | 4 |
| before testing the Sleep Nasal spray- scaled | | | | | | |
| on a 0-10 scale? 0: Not fresh at all - very | | | | | | |
| sleepy-10: Very fresh and ready for the day | | | | | | |
| How fresh/awake did you feel the day after | 6 | 3 | 2 | 4 | 4 | 4 |
| using the Sleep Nasal spray- scaled on a 0-10 | | | | | | |
| scale? 0: Not fresh at all - very sleepy-10: | | | | | | |
| Very fresh and ready for the day | | | | | | |
| How deep a sleep did you feel you have had | 3 | 4 | 3 | 4 | 3 | 2 |
| during the night before testing the Sleep | | | | | | |
| Nasal spray- scaled on a 0-10 scale? 0: No | | | | | | |
| deep sleep-10: Very good and deep a sleep | | | | | | |
| How deep a sleep did you feel you have had | 4 | 4 | 3 | 4 | 4 | 3 |
| during the night during testing the Sleep | | | | | | |
| Nasal spray- scaled on a 0-10 scale? 0: No | | | | | | |
| difference than before not using Sleep Nasal | | | | | | |
| spray-10: Very good and deep a sleep | | | | | | |
| Medication reduced during test period? | NA | NA | NA | NA | NA | NA |

### Example 5

Further nasal formulations with different ratios and concentrations of CBD and CBN were provided according to Example 1.

Appropriate amounts of CBD and CBN were dissolved in hemp oil, essentially free of cannabinoids.

CBD and CBD are provided as “crystalline” or “pure” CBD in powder-form, with a purity of at least 98%, and comprising less than 1.5% (w/w) of CBDV, CBG, and/or CBN, and less than 0.05% THC.

### Formulation D “CBN=CBD” (0.42% CBD and 0.42% CBN)

| | Purity* | Weight | Concentration |
|---|---|---|---|
| Ingredient | (%) | (g) | % (w/w) |
| Hemp oil (comprising 5% | 90 | 12.5 | 16.98 |
| CBD and 5% CBN by | | | |
| weight) | | | |
| Lavender oil - russisch, | 100 | 0.02 | 0.03 |
| Vögele, 00002368 EP grade | | | |
| Sesam Oil (Ölmühle | 100 | 60.1 | 81.64 |
| Hartman) EP grade | | | |
| Vitamin E - Oil - EP | 40 | 1 | 1.36 |
| grade** | | | |
| | Total | 73.62 | 100.00 |

*% active compound in ingredient
**0.4 g Tocopherol equivalent to 1 g Vit. E oil

### Formulation E “10CBD>CBN” (8.49% CBD and 0.85% CBN)

| | Purity* | Weight | Concentration |
|---|---|---|---|
| Ingredient | (%) | (g) | % (w/w) |
| Hemp oil (comprising 50% | 90 | 12.5 | 16.98 |
| CBD and 5% CBN by | | | |
| weight) | | | |
| Lavender oil - russisch, | 100 | 0.02 | 0.03 |
| Vögele, 00002368 EP grade | | | |
| Sesam Oil (Ölmühle | 100 | 60.1 | 81.64 |
| Hartman) EP grade | | | |
| Vitamin E - Oil - EP | 40 | 1 | 1.36 |
| grade** | | | |
| | Total | 73.62 | 100.00 |

*% active compound in ingredient
**0.4 g Tocopherol equivalent to 1 g Vit. E oil

### Formulation F (2.55% CBD)

| | Purity* | Weight | Concentration |
|---|---|---|---|
| Ingredient | (%) | (g) | % (w/w) |
| Hemp oil (comprising 15% | 90 | 12.5 | 16.98 |
| CBD) | | | |
| Lavender oil - russisch, | 100 | 0.02 | 0.03 |
| Vögele, 00002368 EP grade | | | |
| Sesam Oil (Ölmühle | 100 | 60.1 | 81.64 |
| Hartman) EP grade | | | |
| Vitamin E - Oil - EP | 40 | 1 | 1.36 |
| grade** | | | |
| | Total | 73.62 | 100.00 |

*% active compound in ingredient
**0.4 g Tocopherol equivalent to 1 g Vit. E oil

### Formulation G “4CBD>CBN” (3.4% CBD and 0.85% CBN)

| | Purity* | Weight | Concentration |
|---|---|---|---|
| Ingredient | (%) | (g) | % (w/w) |
| Hemp oil (comprising 20% CBD | 90 | 12.5 | 16.98 |
| and 5% CBN) | | | |
| Lavender oil - russisch, Vögele, | 100 | 0.02 | 0.03 |
| 00002368 EP grade | | | |
| Sesam Oil (Ölmühle Hartman) EP | 100 | 60.1 | 81.64 |
| grade | | | |
| Vitamin E - Oil - EP grade** | 40 | 1 | 1.36 |
| | Total | 73.62 | 100.00 |

*% active compound in ingredient
**0.4 g Tocopherol equivalent to 1 g Vit. E oil

### Formulation H “2CBD>CBN” (1.7% CBD and 0.85% CBN)

| | Purity* | Weight | Concentration |
|---|---|---|---|
| Ingredient | (%) | (g) | % (w/w) |
| Hemp oil (comprising 10% | 90 | 12.5 | 16.98 |
| CBD and 5% CBN) | | | |
| Lavender oil - russisch, | 100 | 0.02 | 0.03 |
| Vögele, 00002368 EP grade | | | |
| Sesam Oil (Ölmühle | 100 | 60.1 | 81.64 |
| Hartman) EP grade | | | |
| Vitamin E - Oil - EP | 40 | 1 | 1.36 |
| grade** | | | |
| | Total | 73.62 | 100.00 |

*% active compound in ingredient
**0.4 g Tocopherol equivalent to 1 g Vit. E oil

### Example 6—Formulation Comprising Water and NaCl

### Formulation 1 (0.45 CBD and 0.87% CBN; 0.91% NaCl)

| Raw mat. (RM) | gram | % (w/w) |
|---|---|---|
| CBD | 0.22 | 0.45 |
| CBN | 0.43 | 0.87 |
| Ethanol undenatured, 96.4% | 1 | 2.03 |
| NaCl | 0.45 | 0.91 |
| Water dist. | 44.1 | 89.63 |
| Lavender oil | 1 | 2.03 |
| Emulsifier: Span80 | 0.7 | 1.42 |
| Surfactant: Polysorbate 80 | 1.3 | 2.64 |
| Total | 49.2 | 100 |

### Example 7—Results Formulations D-I

### Results of Tests With Formulations D-H.

| Test person | 1 | 2 | 3 | 4 | 5 |
|---|---|---|---|---|---|
| Male (M) or Female (F) | M | F | F | F | F |
| SD type (see Example 3, incl. | SD: 1, | SD: 1 | SD: 1 | SD: 1 | SD: 1 |
| criteria) | CBN = | 10CBD > | CBD | 4CBD > | 2CBD > |
| Ratio; Formulation | CBD; D | CBN; E | | CBN; G | CBN; H |
| Test period(days) | 3 | 2 | 3 | 2 | 1 |
| How many puffs (spray's per | 2 | 1 | 1 | 1 | 2 |
| nostril) did you apply before | | | | | |
| going to bed? | | | | | |
| How much do the sleep disorder | 3 | 4 | 4 | 5 | 4 |
| effect your life - scaled on a 0- | | | | | |
| 10 scale (0 = no effect; 10 = | | | | | |
| worst ever experienced - always | | | | | |
| sleepy) before testing Sleep | | | | | |
| Nasal Spray? | | | | | |
| How fast did you fall asleep in | B: 60 | B: 60 | B: 90 | B: 90 | B: 60 |
| average | D: 30 | D: 60 | D: 60 | D: 90 | D: 40 |
| before testing Sleep Nasal spray | | | | | |
| (in minutes)? | | | | | |
| How fast did you fall asleep in | | | | | |
| average | | | | | |
| During test period of Sleep | | | | | |
| nasal spray (in minutes)? | | | | | |
| How much coherent sleep, did | B: 3 | B: 3 | B: 2 | B: 3 | B: 3 |
| you get in average before | D: 4 | D: 3 | D: 2 | D: 3 | D: 4 |
| testing Sleep Nasal spray (in | | | | | |
| hours)? | | | | | |
| How much coherent sleep, did | | | | | |
| you get in average During test | | | | | |
| period of Sleep nasal spray (in | | | | | |
| hours)? | | | | | |
| How many times did you wake | B: 3 | B: 3 | B: 3 | B: 4 | B: 2 |
| up during the night | D: 2 | D: 2 | D: 3 | D: 4 | D: 2 |
| before testing Sleep Nasal spray | | | | | |
| (in hours)? | | | | | |
| How many times did you wake | | | | | |
| up during the night | | | | | |
| During testing Sleep Nasal | | | | | |
| spray (in hours)? | | | | | |
| How fresh/awake did you feel | 5 | 4 | 3 | 2 | 4 |
| the day after - before testing the | | | | | |
| Sleep Nasal spray- scaled on a | | | | | |
| 0-10 scale? 0: Not fresh at all - | | | | | |
| very sleepy - 10: Very fresh and | | | | | |
| ready for the day | | | | | |
| How fresh/awake did you feel | 6 | 4 | 3 | 2 | 4 |
| the day after using the Sleep | | | | | |
| Nasal spray- scaled on a 0-10 | | | | | |
| scale? 0: Not fresh at all - very | | | | | |
| sleepy - 10: Very fresh and | | | | | |
| ready for the day | | | | | |
| How deep a sleep did you feel | 4 | 4 | 3 | 3 | 4 |
| you have had during the night | | | | | |
| before testing the Sleep Nasal | | | | | |
| spray- scaled on a 0-10 scale? 0: | | | | | |
| No deep sleep - 10: Very good | | | | | |
| and deep a sleep | | | | | |
| How deep a sleep did you feel | 4 | 4 | 3 | 3 | 4 |
| you have had during the night | | | | | |
| during testing the Sleep Nasal | | | | | |
| spray- scaled on a 0-10 scale? 0: | | | | | |
| No difference than before not | | | | | |
| using Sleep Nasal spray - 10: | | | | | |
| Very good and deep a sleep | | | | | |
| Medication reduced during test | NA | NA | NA | NA | NA |
| period? | | | | | |

### Results of Tests With Formulation I

This test was discontinued, as all participants experienced formulation 1 (0.9% NaCl) as strongly irritating for the nostrils which prolonged the time to fall asleep.

| Test person | 1 | 2 | 3 | 4 |
|---|---|---|---|---|
| Male (M) or Female (F) | Male | Male | Female | Female |
| SD type (see Example 3, incl. criteria) | SD: 1 | SD: 1 | SD: 1 | SD: 1 |
| Test period(days) | 1 | 1 | 1 | 1 |
| How many puffs (spray's per nostril) did you | 1 | 1 | 1 | 1 |
| apply before going to bed? | | | | |
| How much do the sleep disorder effect your life - | 3 | 4 | 5 | 4 |
| scaled on a 0-10 scale (0 = no effect; 10 = worst | | | | |
| ever experienced - always sleepy) before testing | | | | |
| Sleep Nasal Spray? | | | | |
| How fast did you fall asleep in average | B: 15 | B: 90 | B: 90 | B: 30 |
| before testing Sleep Nasal spray (in minutes)? | D: 180 | D: 120 | D: 180 | D: 120 |
| How fast did you fall asleep in average | | | | |
| During test period of Sleep nasal spray (in | | | | |
| minutes)? | | | | |

### Example 8

Polysomnography and/or actigraphy are tests commonly used in the field to analyse sleeping patterns and/or behaviours. They can be used in experiments, when assessing the efficacy of a sleep formulation and/or delivery route presented herein, such as by comparing the sleep patterns/behaviours with or without treatment, or different treatments, such as one or more compositions according to the present invention, with a conventional treatment, optionally including a negative control and/or a placebo.

### Example 9—Provision of CBD by Alcohol Extraction, Distillation and Crystallization

Crystalline CBD can be provided by methods and techniques known in the art, such as by methods disclosed in U.S. Pat. No. 10,413,845 and/or U.S. Pat. No. 10,414,709.

In short, crystalline CBD can be provided from hemp or Cannabis (Cannabis sativa) by a method consisting essentially of:

Extracting hemp or Cannabis with a solvent selected from the group consisting of propanol, isopropanol, butanol, pentanol, hexanol, heptanol, and octanol to produce an extract consisting essentially of an extracted hemp or Cannabis consisting essentially of tetrahydrocannabinol, a terpene, or cannabidiol;

Evaporating the solvent portion of the extract to generate a substantially solvent-free extract comprising CBD;

Distilling the substantially solvent-free extract to isolate the CBD, and

Crystallizing the distilled, isolated CBD to produce a crystallized, isolated CBD.

Often, the crystallized, isolated CBD is subjected to vacuum drying to remove volatile remnants, in particular the solvent used in crystallizing or re-crystallizing, if needed.

In particular, a method comprising extraction with isopropanol and crystallization by the use of heptane, including one or more optional re-crystallization steps, followed by vacuum drying can provide CBD with crystal structure A, i.e. needle like crystals. Furthermore, such a CBD can be very low in undesired compounds, such as terpenes.

GC chromatography or other analytical methods known in the art can be used to monitor the process such as to ensure a high yield and/or a high purity of the desired product.

Concerning the raw material, hemp comprising e.g. 2-3% CBD is dried and ground before extraction with isopropanol, such as food grade isopropanol.

Guidance for choosing the appropriate reaction based on the boiling points or ranges of the different compounds can e.g. be found here: www.nwsci.com/customer/docs/SKUDocs/RMR/Technical%20Data_Extractions_03.28.18.pdf.

CBD with crystal structure A can e.g. be provided from www.enecta.com, and/or following a similar extraction and/or purification protocol as said manufacturer.

### Example 10—Comparison of Nasal Sleep Compositions Formulated With Different Crystalline CBDs

Two nasal sleep compositions are prepared as disclosed herein, such as according to Example 1, 5 and/or 7, the only difference being that the crystalline CBD used in the formulation is either of type A (needle-like crystals; FIG. 1) or type B (bunch/cluster-like; FIG. 2).

Type A crystalline CBD is sourced from Enecta, while type B CBD is sourced from Pharma Hemp. Further details can e.g. be found in the first aspect of the invention, such as Table 1.

When testing both nasal sleep compositions, surprisingly and unexpectedly, it is seen and/or it can be concluded that type A CBD is significantly more active than type B CBD.

1-53. (canceled)

54. Composition comprising:
i. 5-30%, 10-20%, or ˜16% hemp oil;
ii. (a) 0.1-5.0%, 0.2-2.0%, or ˜0.4% cannabidiol (CBD);
(b) 0.1-5.0%, 0.2-2.0%, or ˜0.8% cannabinol (CBN); or
(c) 0.1-5.0%, 0.2-2.0%, or ˜0.4% cannabidiol (CBD), and 0.1-5.0%, 0.2-2.0%, or ˜0.8% cannabinol (CBN);
iii. 0.01-1.0%, 0.02-0.5%, or ˜0.03% lavender oil;
iv. 30-95%, 50-90%, or ˜82% sesame oil; and
0.1-5.0%, 0.2-1.0%, or ˜0.55% vitamin E.

55. Composition according to claim 54 formulated as a nasal spray.

56. Composition according to claim 54 formulated as a nasal spray for intra-nasal application.

57. Composition according to claim 54, comprising CBN and CBD, wherein the CBN:CBD ratio by weight is greater than 1.

58. Composition according to claim 57, wherein the CBN:CBD ratio in the range of 5:1 to 1:1, 4:1 to 1:1, 3:1 to 1: 1, 2.5:1 to 1:1, or around 2:1.

59. Composition according to claim 54, wherein the composition comprises one or more further cannabinoid(s) selected from one or more of: THC (tetrahydrocannabinol), THCA (tetrahydrocannabinolic acid), CBDA (cannabidiolic acid), CBG (cannabigerol), CBC (cannabichromene), CBL (cannabicyclol), CBV (cannabivarin), THCV (tetrahydrocannabivarin), THCP (tetrahydrocannabiphorol), CBDV (cannabidivarin), CBCV (cannabichromevarin), CBGV (cannabigerovarin), CBGM (cannabigerol monomethyl ether), CBE (cannabielsoin), CBT (cannabicitran), or any combination(s) thereof.

60. Composition according to claim 54, wherein the composition comprises less than 1.5, 1.0, 0.5, 0.2, or 0.1% (w/w) of one or more of: THC, THCA, CBDA, CBG, CBC, CB, CBV, THCV, THCP, CBDV, CBCV, CBGV, CBGM, CBE, CBT, or any combination(s) thereof.

61. Composition according to claim 54, wherein the hemp oil comprises less than 1.5, 1.0, 0.5, 0.2, or 0.1% (w/w) of one or more further cannabinoid(s) selected from: THC, THCA, CBDA, CBG, CBC, CB, CBV, THCV, THCP, CBDV, CBCV, CBGV, CBGM, CBE, CBT, or any combination(s) thereof.

62. Composition according to claim 54, wherein the composition does not comprise one or more further cannabinoid(s) selected from: THC, THCA, CBDA, CBG, CBC, CB, CBV, THCV, THCP, CBDV, CBCV, CBGV, CBGM, CBE, CBT, or any combination(s) thereof, in a physiologically active amount.

63. Composition according to claim 59, wherein the composition comprises one or more further cannabinoid(s) selected from:
≤0.2% or ≤0.05% CBDV by weight;
≤0.2% or ≤0.05% CBDA by weight;
≤0.5% or ≤0.025% CBG by weight;
≤0.05% or ≤0.020% THC by weight; or
any combination(s) thereof.

64. Composition according to claim 59, wherein the further cannabinoid(s) is/are psychoactive cannabinoid(s) or non-psychoactive cannabinoid(s), said non-psychoactive cannabinoid(s) being selected from one or more of: THCA, CBDA, CBG, CBC, CBL, CBV, THCP, CBDV, CBCV, CBGV, CBGM, CBE, CBT, cannabinoid(s) not binding to a CB1 receptor, or any combination(s) thereof.

65. Composition according to claim 54, wherein the composition comprises less than 0.1% (w/w) sodium chloride.

66. Composition according to claim 54, wherein the composition comprises less than 1.0 (w/w) water.

67. Composition according to claim 59, wherein:
the hemp oil used in the provision of the composition comprises ≤0.2% or ≤0.05% CBDV by weight; ≤0.2% or ≤0.05% CBDA by weight; ≤0.5% or ≤0.025% CBG by weight; and <0.05% or ≤0.020% THC by weight;
the lavender oil used in the provision of the composition is produced by steam distillation of Lavandula angustifolia flowers (CAS no. 8000-28-0, and comprises one or more of: VOC-CH content of ˜1% (by weight); ˜0.05% coumarin, CAS No. 91-64-5; ˜0.40% geraniol, CAS No. 106-21-1; ˜0.5% D-limonene, CAS no. 5989-27-5; and ˜30% Linalool, CAS no. 78-70-6; including any combination(s) thereof; and
the sesame oil used in the provision of the composition is a refined oil with the following specifications: acid ≤0.5, peroxide value ≤10.0, unsaponified matter ≤2% (w/w), alkaline substances ≤0.1, water ≤0.1%; the sesame oil further comprising the following triglyceride composition by weight: LLL 7-19% LLL, 13-30% OLL, 5-9% PLL, 12-23% OOL, 6-14% POL, 5-16% 000, 2-8% SOL, and 2-10% POO; the fatty acid radicals are designated as linoleic (L), oleic (O), palmitic (P), and stearic (S).

68. Composition according to claim 54, wherein the CBD used in the provision of the CBD-comprising composition is crystalline; and wherein the CBD crystals are selected from one or more of:
needle-like crystals;
crystals not provided by an extraction method comprising critical CO2 extraction; and
crystals provided by a method comprising extraction with a C3-C4 alcohol, and one or more crystallisations steps with a C6-C8 alcohol; or
any combination thereof.

69. A method for providing a composition for nasal application according to claim 54, comprising the steps of:
i. providing hemp oil comprising CBD, CBN or a combination thereof;
ii. providing lavender oil, sesame oil and vitamin E (e.g. vitamin E oil); and
iii. mixing the hemp oil from step (i) with the ingredients from step (ii).

70. A method of treatment of a sleep-related condition in a subject comprising:
administering the composition of claim 54 to the subject; and
treating the sleep-related condition in the subject,
wherein said sleep-related condition is selected from one or more of:
i. Insomnia
ii. Snoring
iii. Obstructive Sleep Apnea
iv. Sleep Hypoventilation
v. Restless Legs Syndrome
vi. Bruxism
vii. Narcolepsy
viii. Sleep talking, sleep walking and/or other automatic behaviors
ix. Nightmares and/or night terrors; and
X. Rapid eye movement behavior disorder;
xi. Jet lag;
xii. Difficulties in falling asleep under unusual circumstances;
xiii. Anxiety;
xiv Parkinson;
xv. Multiple sclerosis (MS);
xvi. Muscle spasm;
xvii. Depression;
xviii. Alzheimer;
xix. Epilepsy;
xx. Pain;
xxi. Neurological condition(s) requiring a protective effect; or
any combination(s) thereof.

71. Method of treatment according to claim 70 wherein the composition is the composition of claim 56 and the composition is administered to the subject by intra-nasal application.

72. Method of treatment according to claim 71, wherein the composition is administered to the subject according to a dosage regimen comprising one or more of:
50-350 μl, 100-250 μl, or around 160 μl in each nostril;
a total of 0.5-5, 1.5-3.5, or around 2.8 mg CBN per application;
a total of 0.5-4, 0.8-1.8, or around 1.4 mg CBD per application; or
any combination(s) thereof.

73. A kit comprising a receptacle, wherein said receptacle is suitable for intra nasal application of the composition according to claim 2 to a subject, said receptacle providing light or UV-protection to the composition; the receptacle being a nasal pump spray bottle, said nasal pump spray bottle being adapted to administer 50-350 μl, 100-250 μl, or around 160 μl per puff to a nostril.
